(19) United States
(12) Patent Application Publication
(10) Pub. No.: US 2007/0141661 A1
(43) Pub. Date: Jun. 21, 2007

(54) Cell extract for high-functioned cell-free protein synthesis and method of preparing the extract

(76) Inventors: Yaeta Endo, Ehime (JP); Tomio Ogasawara, Ehime (JP)

Correspondence Address: JHK LAW, P.O. BOX 1078, LA CANADA, CA, 91012-1078, US

(21) Appl. No.: 10/596,538

(22) PCT Filed: Dec. 17, 2004
(86) PCT No.: PCT/JP04/18928

§ 371 Date: Jul. 10, 2006

(30) Foreign Application Priority Data
Dec. 26, 2003 (JP) 2003-434080

(51) Int. Cl. C12P 21/06; C12N 9/64

(52) U.S. Cl. 435/68.1; 435/226

(57) ABSTRACT

The subjects of the present invention are to prepare a highly functionalized cell extract for cell-free protein synthesis and to specify and eliminate inhibitors and unstable substances present in conventional and various cell extracts for cell-free protein synthesis. Also provided is a method for preparing the cell extract for use in a cell-free protein synthesis means, wherein ATP-mediated phosphorylation pathway of sugar present in the cell extract is controlled. In particular, the control is introducing at least one selected from the following: 1) removing monosaccharides, 2) removing phosphorylated sugars, 3) controlling production of monosaccharides from polysaccharides, and 4) controlling production of phosphorylated sugars from monosaccharides.

### TECHNICAL FIELD

This application claims the priority of Japanese Patent Application No. 2003-434080, which is incorporated herein by reference.

The present invention relates to a highly functionalized cell extract for use in cell-free protein synthesis, and a method for preparation thereof and the like. In particular, it relates to a highly functionalized cell extract, wherein a cell-intrinsic inducible system of inhibition of protein synthesis, which is present in a cell extract for cell-free protein synthesis, is blocked, and a method of preparation thereof and the like. More particularly, it relates to at least a method of eliminating translational inhibitors for genetic information, present in a cell extract for cell-free protein synthesis, wherein metabolic pathway of sugar phosphorylation is controlled.

### BACKGROUND ART

Concerning a cell-free protein synthesis system, since a ground cell fluid still retained a protein synthesis capacity was reported forty years ago, various methods have been developed and cell extracts such as E. coli, wheat embryo, rabbit reticulocyte-derived and insect-derived have currently and broadly been used in protein synthesis and the like. Translation rates in a cell-free system were almost the same as those in vivo, which were 10 peptide bonds/sec, exerting excellent reaction properties in speed and translation accuracy, but durations of synthesis were short in every cell-free system and resulting yields were significantly small such that they were from a few to tens μg/ml reaction volume equivalent to 1/100 to 1/1000 of those of living cells, thus they were not practical methods for synthesizing proteins.

Though the worst drawback of conventional cell-free protein synthesis systems is their extremely low synthesis efficiency, the cause of that has never been squarely researched. That is because it was a common sense in the biochemistry field that the activity of a cell extract obtained by physically grinding cells and prepared in an artificial buffer is low.

Based on findings obtained from previous researches on ribosome inactivating-toxins, the inventors have already revealed that a phenomenon of an extreme decrease in the activity of protein synthesis seen in a cell-free protein synthesis system using a wheat embryo extract solution is resulted from the fact that a programmed mechanism of ribosomal self-inactivation (apoptosis mechanism) which is originally installed in a cell as a defense mechanism against pathogenic microorganisms is switched on by a trigger of grinding embryos. Then, we demonstrated that the protein synthesis reaction of a wheat embryo extract solution prepared by our novel method exerted high protein synthesis properties over a long period, wherein the method was to eliminate a group of protein synthesis inhibitory factors such as activities of tritin, thionine, RNase, DNase and proteinase from embryo tissues, wherein the factors are localized to seed endosperm and contaminating the extract during embryo-isolating operation (non-patent document No. 1) (patent document No. 1).

[nonpatent document No. 1] Madin, K. etal., Proc. Natl. Acad. Sci. USA, 97, 559-564 (2000)

[patent document No. 1] Japanese Patent Application Laid-open No. 2000-236896

### DISCLOSURE OF THE INVENTION

### Problem to be Solved by the Invention

However, still in a wheat embryo extract solution prepared by the above-described method, such as in a fraction of supernatant obtained by centrifugation at 30,000 g (S-30), it is believed that there are metabolic pathways such as embryo tissue cell-intrinsic glycolytic pathway and a mechanism of controlling translation reaction. Their phenomena and biochemical facts have not been known so far, but it is suspected that at the time of cell-free protein synthesis reaction, those reaction pathways start to operate and along with it, protein synthesis reactions receive negative effects, resulting in not a sufficient synthetic yield. Therefore, by demonstrating the mechanisms of start of intrinsic metabolic pathways and presence of controlling translation reaction which works with that start, and blocking the mechanisms, properties of already successfully developed synthesis methods of wheat embryo cell-free protein can further be expected to improve.

Further, conventional cell extracts for cell-free protein synthesis had a problem in preservation due to the fact that the addition of a solution containing substances necessary for protein synthesis such as amino acids, energy sources and ions might affect intrinsic factors. Thereby, a solution containing a cell extract and that containing energy sources and the like were separately provided, so that an experimenter had to mix them along with a translation template in each case of experiments. In addition, the whole experimental operation was made complicated because of a need for low-temperature operation or the like, often causing a failure in protein synthesis. Further the method for providing a reagent for such cell-free protein synthetic reaction was not suitable for an encompassing synthesis of proteins involving a larger number of genes, and solving the drawbacks of such complications had been the biggest subject for considering the future robotization.

Further, the inventors have found that the activity of protein synthesis of a cell extract is significantly facilitated by the removal of low molecular substances (sometimes referred to as low molecular protein synthesis inhibitors) from above wheat embryo extract solution through the dialysis of said extract solution using a regenerated cellulose membrane with a molecular weight cutoff of from 12,000 to 14,000 Da (Patent Publication WO 03/064672). It appears that this is supporting the postulation described above.

Therefore, the subjects of the present invention are the preparation of a highly functionalized cell extract for cell-free protein synthesis, and the establishment of a cell-free protein synthesis method having higher functionalities by biochemically confirming and verifying the phenomena of inhibition and destabilization of a protein synthesis system involving enzymes in metabolic pathways and intrinsic to conventional wheat embryo cell extracts for cell-free protein synthesis, and specifying and eliminating the related substances.

### Means for Solving the Problem

Generally, as a cell extract for use in cell-free protein synthesis, a cell extract obtained by eliminating low molecules with a molecular sieve such as Sephadex G25 from S-30 fraction which has been obtained by centrifugation at 30,000×gis used. The purposes of these operations are to previously eliminate intrinsic low molecular substances having unclear concentrations and to remove homogenized solution-derived potassium acetate and calcium chloride at high concentration to optimize the concentrations of components necessary for protein synthesis in synthetic reaction pathway such as ions, amino acids and nucleotides. The inventors speculated there still must have been intrinsic protein synthesis inhibitors in a conventional cell extract obtained by such a method. Therefore, we further thoroughly eliminated low molecular substances having weights less than 10,000 Da from a cell extract obtained by a conventional method, by using an ultrafiltration membrane. A cell extract for cell-free protein synthesis from which low molecular substances had been thoroughly eliminated exerted a high protein synthesis capacity. Moreover, adding a filtrate obtained through an ultrafiltration membrane treatment to a highly functionalized cell extract for cell-free protein synthesis in an equivalent amount generated phenomena of inhibition and destabilization in protein synthesis. From these, the inventors confirmed that intrinsic inhibitors inhibiting protein synthesis were present in a conventional cell extract for cell-free protein synthesis.

To identify these intrinsic inhibitors, we spread out the filtrate obtained through an ultrafiltration membrane treatment on a thin layer chromatography, and specified raffinose, the mixture of sucrose and glucose and xylose. Then, we found that a fraction containing inhibitors of interest had especially high glucose content. We confirmed glucose and sucrose led to the phenomena of strong inhibition and destabilization of protein synthesis when added to a cell extract for cell-free protein synthesis.

In addition, the inventors focused on a phenomenon of a remarkable decrease in ATP in a cell-free protein synthesis system, set up hypothesis of a sugar phosphorylation reaction generated by the reaction with sugar in ATP, and examined the effects of various sugar compounds on a cell-free protein synthesis system. In other words, we added glucose, fructose, galactose, phosphorylated glucose, phosphorylated fructose and the like to a cell extract for cell-free protein synthesis whose high functionalities had been confirmed by a high level purification, and those effects are examined. As a result, every sugar induced a phenomenon of remarkable decrease in ATP and also exhibited a strong inhibition of protein synthesis. In particular, glucose, fructose, and phosphorylated products of them had extremely strong inhibitory effects on cell-free protein synthesis. In an experiment of adding L-glucose which is an optical isomer can not be recognized and metabolized by enzymes of an organism, the phenomena of decrease in ATP and inhibition of protein synthesis as well as destabilization of a translation reaction pathway were not generated.

Then, the present inventors experimentally verified the following facts: 1) in a cell extract for cell-free protein synthesis, an embryo-derived glycolytic and enzymatic pathway, which degrades a hexose such as glucose, exists and functions, 2) phosphorylation of sugar, which consumes ATP, occurs through the catalysis of phosphorylation enzymes (hexokinase and glucokinase) and using as a substrate glucose produced by hydrolysis of starch, 3) by the reaction with a high concentration of substrate of glucose present in an embryo extract solution, a large amount of ATP is consumed, which surpasses the production capability of ATP-regenerator of phosphocreatine and creatine kinase, resulting in the state wherein ATP concentration is remarkably decreased in a protein synthetic reaction pathway, 4) protein synthesis inhibition and destabilization of synthesis system are not caused by the increase in AMP concentration generated by the consumption of ATP and therefore the increase in GMP concentration accompanied by the termination of GTP regeneration (i.e. byproducts), but by allowing the decrease in ATP concentration to become a trigger, starting the negative regulator of protein synthesis, and inactivating any of factors in protein synthesis reaction. Recently, phosphorylation reaction in α-subunit of translation initiation factor (eIF2) and a physiological mechanism of translation reaction control (inhibition) by a phosphorylation enzyme (pPKR) which catalyzes above phosphorylation reaction in plant, were reported (J. Biol. Chem. vol. 271, pp. 4539-4544 (1996), Biochemistry vol. 39, pp. 7521-7530 (2000)). A possibility of close relation between a phenomenon which is mediated by ATP found in the present invention that is: phosphorylation with enzymes in glycolytic pathway in a sugar such as glucose→decrease in ATP concentration→inhibition of protein synthesis reaction; and phosphorylation mechanism of eIF2 with pPKR, can be considered. In the present invention, we examined means for preparing a highly functionalized extract for cell-free protein synthesis on the basis of such findings, and completed the present invention by achieving the control of a metabolic pathway of sugar phosphorylation intrinsic to an extract for cell-free protein synthesis.

Therefore, the present invention consists of the following:
“1. A method for preparing a cell extract for use in a cell-free protein synthesis means, comprising elimination of a cell-derived mechanism for inhibition of translation.
2. The method according to previous 1, wherein the elimination of a cell-derived mechanism for inhibition of translation is provided by controlling ATP-mediated sugar phosphorylation pathway.
3. The method according to previous 1 or 2, wherein the cell-derived mechanism for inhibition of translation is an embryo cell-intrinsic inducible system of inhibition of protein synthesis.
4. The method according to any one of the previous 1 to 3, wherein a source of the cell extract is a wheat embryo extract in which contaminating endosperm components and low molecular protein synthesis inhibitors are substantially removed.
5. The method according to previous 1 or 2, wherein a source of the cell extract is E. coli, rabbit reticulocyte or insect cell extract.
6. The method according to previous 2, wherein the ATP-mediated sugar phosphorylation pathway is controlled by introducing at least one step selected from the followings:
   1) removing monosaccharides,
   2) removing phosphorylated sugars,
   3) controlling production of monosaccharides from polysaccharides, and
   4) controlling production of phosphorylated sugars from monosaccharides.
7. The method according to previous 6, wherein, in removing monosaccharides, the monosaccharide is a hexose.
8. The method according to previous 6, wherein the phosphorylated sugar is at least one selected from glucose 1-phosphate, fructose 1-phosphate, galactose 1-phosphate, glucose 1,6-diphosphate, fructose 1,6-diphosphate, galactose 1,6-diphosphate in removing phosphorylated sugars.
9. The method according to previous 6, wherein the monosaccharides and/or the phosphorylated sugars are removed by fractional elimination of molecular weight carried out by gel filtration and/or with an ultrafiltration membrane.
10. The method according to previous 9, wherein the fractional elimination of molecular weight carried out by gel filtration and/or with an ultrafiltration membrane is repeated multiple times.
11. The method according to previous 6, wherein the production of monosaccharides from polysaccharides is controlled by controlling production of glucose from starch.
12. The method according to previous 11, wherein the production of monosaccharides from polysaccharides is controlled by introducing at least one step selected from the followings:
   1) removing or inactivating glycolytic enzymes,
   2) eliminating polysaccharides and/or oligosaccharides, and/or disaccharides, and
   3) adding a glycolytic enzyme inhibitor.
13. The method according to previous 12, wherein a glycolytic enzyme is removed or inactivated by removing a complex between said glycolytic enzyme and calcium after forming the complex.
14. A method for preparing cell extract, wherein removal of a cell-derived glycolytic enzyme is introduced by adding at least one selected from bentonite, activated carbon, silica gel, Sephadex and sea sand to said cell extract as a precipitation auxiliary agent.
15. The method according to previous 6, wherein the production of phosphorylated sugars from monosaccharides is controlled by introducing at least one step selected from the followings:

1) introducing an inhibitor against a sugar phosphorylation enzyme,
2) removing or inactivating an sugar phosphorylation enzyme,
3) eliminating said production from glucose metabolic pathway by enzymatic degradation of a hexose,
4) inhibiting an enzymatic reaction of sugar phosphorylation by chemical or enzymological modification of a hexose,
5) enzymatically and/or chemically alternating and/or modifying a phosphorylation site of the sugar, so that a phosphate group cannot bind to said phosphorylation site of the sugar.
16. The method according to previous 7, wherein the hexose is glucose.
17. The method according to previous 16, wherein a concentration of glucose in the cell extract is 10 mM or less when a concentration of the cell extract is 200 OD 260 nm.
18. The method according to previous 16, wherein a concentration of glucose in the cell extract is 6 mM or less when a concentration of the cell extract is 200 OD 260 nm.
19. The cell extract for use in a cell-free protein synthesis means prepared by the method according to any one of the previous 1 to 18.
20. A cell extract for use in a cell-free protein synthesis means, wherein ATP-mediated sugar phosphorylation pathway is controlled.
21. The cell extract according to previous 20, wherein the ATP-mediated sugar phosphorylation pathway is controlled by introducing at least one step selected from the followings:
   1) substantially removing or inactivating phosphorylated sugars,
   2) substantially removing polysaccharides, oligosaccharides, disaccharides and monosaccharides,
   3) substantially removing or inactivating glycolytic enzymes,
   4) adding a glycolytic enzyme inhibitor,
   5) substantially removing or inactivating phosphorylation enzymes,
   6) adding a phosphorylation enzyme inhibitor,
   7) enzymatically and/or chemically alternating and/or modifying a phosphorylation site of the sugar, so that a phosphate group cannot bind to said phosphorylation site of the sugar.
22. A cell-free protein synthesis method using the cell extract according to any one of previous 19 to 21.
23. A use of cell-free protein synthesis system using the cell extract according to any one of previous 19 to 21.
24. A reagent kit for use in a cell-free protein synthesis system comprising the cell extract according to any one of previous 19 to 21.”

### Effects of Invention

A cell extract for cell-free protein synthesis of the present invention was produced by a novel method, and its function reached an unprecedented stability and a high functionality in a cell-free protein synthesis capacity.

### DESCRIPTION OF THE PREFERRED EMBODIMENT

Cell extract for use in the preparation of a cell extract for cell-free protein synthesis of the present invention maybe any one as long as it has a protein synthesis capacity in a cell-free protein synthesis system. Herein, the cell-free protein synthesis system means an in vitro method for synthesizing proteins by extracting components containing ribosome, which is a protein translation apparatus present in a cell, and the like from an organism, and adding a transcription or translation template, nucleic acids as substrate, amino acids, energy sources, various ions, buffer, and other effective factors to this extract solution. For that system, there are the one using RNA as template (hereinafter sometimes referred to as “cell-free translation system”) and the one using DNA and further added with enzymes necessary for transcription such as RNA polymerase before reaction (hereinafter sometimes referred to as “cell-free transcription/translation system”). The cell-free protein synthesis system in the present invention includes both the cell-free translation system and the cell-free transcription/translation system described above.

As a cell extract for use in the present invention, known cell extracts from, specifically, E. coli, embryo of plant seed, rabbit reticulocyte, insect-derived cell and the like can be used. A cell extract can be commercially available one or prepared by a method known per se, and in particular, E. coli extract solution can be prepared in accordance with the method described in Pratt, J. M. et al., Transcription and Translation, Hames, 179-209, B. D. & Higgins, S. J., eds, IRL Press, Oxford (1984).

As a commercially available cell extract, those derived from E. coli include, E. coli S30 extract system (supplied by Promega) and RTS 500 Rapid Translation System (supplied by Roche), etc., those derived from rabbit reticulocyte include Rabbit Reticulocyte Lysate System (supplied by Promega), etc., and further those derived from wheat embryo include PROTEIOS™ (supplied by TOYOBO), etc. Among them, an extract solution from the embryo of plant seed may be preferably used, and for the plant seed, gramineous plants such as wheat, barley, rice and corn may be preferable. As a cell extract of the present invention, among them, the one using wheat embryo extract solution may be preferable. Further, among insect-derived cells, a cell extract solution derived from silkworm and the like can be used.

As a method for making wheat embryo extract solution, methods described in, for example, Johnston, F. B. et al., Nature, 179, 160-161 (1957); or Erickson, A. H. et al., (1996) Meth. In Enzymol., 96, 38-50 can be used.

In the present invention, from such a previously known cell extract for cell-free protein synthesis, inhibitors for cell-free protein synthesis, which could not be confirmed and removed by a conventional method, are eliminated. In other words, the elimination of contaminants from wheat seed endosperms was the principle and means of conventional methods. However, the present method consists of blocking both the function of enzyme group intrinsic to an embryo tissue cell and a function relating to negative control of translation reaction, namely embryo cell-intrinsic inducible system of inhibition of protein synthesis, thus the method is characterized by controlling a phosphorylation pathway mediated by ATP of sugar present with the extract. This pathway is a biologically important metabolic pathway and complicatedly interacting with the control of a glycolytic pathway associated with the energy metabolism of cell and the synthesis of ribose which is a component of nucleic acids, finally resulting in the inhibition of cell-free protein synthesis. Therefore, we found metabolic pathways from polysaccharides to oligosaccharides and disaccharides, and further to monosaccharides, and the production of phosphorylated products mediated by ATP in monosaccharides, were control elements important in cell-free protein synthesis. The control of these pathways leads to a substantial improvement in the protein synthesis function of a cell extract for cell-free protein synthesis.

Further, even in E. coli and reticulocyte, like a higher plant such as cells from plant tissues, a glycolytic pathway associated with the energy metabolism of cell and the synthesis of ribose which is a component of nucleic acids is generally present. In particular, the glycolytic pathway is active in E. coli and reticulocyte. Therefore, metabolic pathways from polysaccharides to oligosaccharides and disaccharides and further to monosaccharides, and the production of phosphorylated products mediated by ATP in monosaccharides, are important control elements in cell-free protein synthesis derived from E. coli and rabbit reticulocyte, thus it is believed that the control of these pathways lead to a substantial improvement in protein synthesis function.

Eliminating the mechanism of controlling translation in the present invention can be achieved by controlling ATP-mediated sugar phosphorylation pathway, and controlling ATP-mediated sugar phosphorylation pathway can be achieved by introducing at least one means like the followings:
1) controlling production of monosaccharides from polysaccharides,
2) removing monosaccharides,
3) removing phosphorylated sugars, and
4) controlling production of phosphorylated sugars from monosaccharides.

Controlling the production of monosaccharides from polysaccharides means to control a reaction pathway from starch of polysaccharides to glucose via oligosaccharides or disaccharides or that to monosaccharides like fructose, so that it means to eliminate the continuous production of monosaccharides in a cell extract. This elimination can be achieved by substantially removing polysaccharides, oligosaccharides and disaccharides from a cell extract. Alternatively, it can be achieved by removing or inactivating glycolytic enzymes, or adding those inhibitors.

Polysaccharides and oligosaccharides and disaccharides can be removed employing a method well known per se such as molecular weight fractionation, affinity chromatography or an inorganic adsorbate treatment. Herein, examples of polysaccharides include starch and amylose, and examples of oligosaccharides and disaccharides include sucrose and malt sugar.

For removing glycolytic enzymes, a widely known means for purifying glycolytic enzymes such as well known affinity chromatography using an antibody and Ion Exchange Chromatography can be employed. Further, it can also be removed by forming a complex between a glycolytic enzyme and calcium by centrifugation. On the occasion of centrifugation, as a precipitation auxiliary agent, a carrier for chromatography such as bentonite, activated carbon, silica gel and Sephadex; or an inorganic carrier such as sea sand will be added. The addition of these precipitation auxiliary agents substantially prevents a supernatant fraction after centrifugation from being contaminated with the precipitate. If a precipitation auxiliary agent is not added on centrifugation, an insoluble slurry would be generated on the upper part of the precipitate, and an extract solution prepared from an S-30 fraction contaminated with it would show a lower activity of protein synthesis. Therefore, on collecting an S-30 fraction from a centrifuge tube after centrifugation, a meticulous attention is needed to avoid contamination. Herein, examples of glycolytic enzymes include enzymes to degrade polysaccharides, oligosaccharides and disaccharides, such as amylase, maltase and glycosidase.

The inactivation is usually carried out by selecting non-reactive conditions contrary to the optimum reaction conditions such as pHs and temperatures of each enzyme. Alternatively, the inactivation can be achieved by using selected conditions of temperatures and/or pHs for selected treatment periods, while considering the general inactivation conditions of enzymes and the other effects on cell-free protein synthesis system.

For an inhibitor against a glycolytic enzyme, widely known substances can be applied. For the amount of addition of it, the conditions which are effective in the inhibition of glycolytic enzymes but otherwise ignorably ineffective on a cell-free protein synthesis system are selected by repeating experiments.

Removing monosaccharides means to substantially eliminate monosaccharides, in particular hexose from a cell extract. Examples of hexoses include glucose, galactose and fructose. The removal can be conducted employing a method well known per se such as molecular weight fractionation, affinity chromatography and an inorganic adsorbate treatment.

As it was found that the existing cell extract for cell-free protein synthesis was contaminated with phosphorylated products of monosaccharides, which themselves had a strong inhibitory capacity against cell-free protein synthesis, removing phosphorylated sugars means to substantially eliminate them from the cell extract. As phosphorylated sugars, for example, glucose 1-phosphate, fructose 1-phosphate, galactose 1-phosphate, glucose 1,6-diphosphate, fructose 1,6-diphosphate, galactose 1,6-diphosphate and the like may be mentioned. They can be removed employing a method well known per se, such as molecular weight fractionation, affinity chromatography or an inorganic adsorbate treatment.

The removal of monosaccharides and phosphorylated sugars can be eliminated to some extent with a molecular sieve such as Sephadex G25 which is usually used in the preparation of a cell extract for cell-free protein synthesis. However, to removing monosaccharides and phosphorylated sugars more efficiently, a thorough fractionation by further gelfiltration, filtration with ultrafiltration membrane or the like is desired, for example, fractionation eliminating low molecules using Amicon Ultra-15 centrifugal filter device with molecular weight cutoff of 10,000 Da, 15 ml, 10K NMWL (supplied by MILLIPORE) may be mentioned. Further, it is desired to repeat this fractional operation multiple times. The specific number of multiple times is from 1 to 10, preferably from 2 to 9, more preferably from 3 to 8, and most preferably from 4 to 7.

Further, inactivating phosphorylated sugars means to not allow a phosphorylated sugar to generate further a phosphorylation activity. These inactivation can be carried out by an enzyme reaction well known per se and the like.

Controlling production of phosphorylated sugars from monosaccharides means to control a system in which monosaccharides, in particular, hexose are to be phosphorylated in a cell extract, and substantially eliminate the production of phosphorylated sugars. For that purpose, there are following means: substantially removing monosaccharides, inactivating sugar phosphorylation enzymes, removing sugar phosphorylation enzymes, and/or adding a sugar phosphorylation enzyme inhibitor and the like. Substantially removing monosaccharides is as described above. The inactivation of sugar phosphorylation enzymes is usually carried out by selecting non-reactive conditions contrary to the optimum reaction conditions such as pHs and temperatures of each sugar phosphorylation enzyme. Alternatively, the inactivation can be achieved by using selected conditions of temperatures and/or pHs for selected treatment periods, while considering the general inactivation conditions of each sugar phosphorylation enzyme and the other effects on cell-free protein synthesis system. Further, the inactivation can also be achieved using antibodies specific for these enzymes.

For each inhibitor against sugar phosphorylation enzyme, widely known substances can be applied. For the amount of addition of it, the conditions which are effective in the inhibition of each sugar phosphorylation enzyme but otherwise ignorably ineffective on a cell-free protein synthesis system are selected by repeating experiments. Herein, as a sugar phosphorylation enzyme, hexokinase, in particular, glucokinase, fructokinase and the like can be mentioned.

The control of phosphorylation of sugars can also be achieved by enzymatically and/or chemically modifying and altering a phosphorylation site of sugar. For example, a method by oxidizing an OH group at position 6 of glucose using glucoseoxidase may be mentioned.

As the best cell extract of the present invention is a wheat embryo extract wherein endosperm components of wheat seed and metabolic substances such as glucose providing inhibitory effects on synthesizing proteins in an embryo tissue cell are substantially removed, the preparation method of source material will be explained below using it as example.

As an embryo part is usually very small, it is preferred to remove parts other than embryo as much as possible to collect embryo efficiently. Usually at first, by applying a mechanical force to plant seeds, a mixture containing embryo, endosperm homogenate and seed coat homogenate is obtained, then the crude embryo fraction (a mixture containing embryo as a principal component and endosperm homogenate and seed coat homogenate) is obtained from the mixture by removing endosperm homogenate and seed coat homogenate and the like. A force applied to the plant seed may be at a certain level enough to separate embryo from plant seed. In particular, the mixture containing embryo and endosperm homogenate and seed coat homogenate will be obtained by grinding plant seeds using a usually known grinding apparatus.

Usually plant seeds can be ground using a widely known grinding apparatus, and a grinding apparatus of type applying an impulse against the material to grind, such as pin mill and hammer mill is preferred. The level of grinding may be selected as appropriate depending on the size of plant seed embryo to use, for example, in the case of wheat seed, it will usually be ground to the size with the maximum length of 4 mm or less, and preferably 2 mm or less. In addition, a dry grinding is preferred.

Then, a crude embryo fraction is obtained from the resulting ground plant seed using a usually known sorting apparatus, for example, sieve. For example, in the case of wheat seed, a crude embryo fraction passing the mesh size from 0.5 mm to 2.0 mm, and preferably from 0.7 mm to 1.4 mm is usually obtained. Further, according to need, seed coat, endosperm, debris and the like contained in the resulting crude embryo fraction may be removed using a wind power or electrostatic power.

Further, a crude embryo fraction can also be obtained by a method using the difference of specific gravities between embryo or seed coat and endosperm, for example, by heavy medium separation. To obtain a crude embryo fraction containing embryo in a higher amount, aplurality of methods described above may be combined. Further, embryo is sorted out, for example, visually or using a color sorter or the like from the resulting crude embryo fraction.

Usually, it is preferred to subject thus obtained embryo fraction to further washing for purifying embryo because the endosperm component sometimes adheres to it. In washing, an embryo fraction is dispersed or suspended in water or an aqueous solution, which is cooled to usually 10° C. or less, and preferably 4° C. or less, in particular in an aqueous solution containing a surfactant, and washed until wash solution becomes clear. Further, it is more preferred to disperse or suspend an embryo fraction in an aqueous solution containing a surfactant and wash it until wash solution becomes clear, usually at 10° C. or less, and preferably 4° C. or less. As a surfactant, nonionic one is preferred, and as long as nonionic surfactant, a broad range of it can be used. In particular, for example, preferable examples include Brij, which is a polyoxyethylene derivative, Triton, Nonidet P40, and Tween. Among them, Nonidet P40 is the most preferred. These nonionic surfactants may be used at a concentration high enough to remove endosperm component but not negatively affect the protein synthesis activity of embryo component, for example, they can be used at a concentration of 0.5%. About washing, either of or both of treatments with water or an aqueous solution and with surfactant may be performed. Further, these washing may be performed with sonication.

In the present invention, a wheat embryo extract solution for cell-free protein synthesis is obtained as indicated above by grinding plant seeds, sorting out plant embryo from the resulting ground product, then washing the plant embryo, fragmenting the obtained and intact (or having germinability) embryo (preferably in the presence of extractant), then separating the resulting wheat embryo extract solution, and further purifying the solution.

As an extractant, a solution containing buffer, potassium ion, magnesium ion and/or an oxidation inhibitor against thiol group in water can be used. Further, according to need, calcium ion, L-amino acid and the like may further be added. For example, a solution containing N-2-hydroxyethyl piperazine-N′-2-ethanesulfonic acid (HEPES)-KOH, potassium acetate, magnesium acetate, L-amino acid and/or dithiothreitol; and a partially altered solution from the one in the method by Patterson et al. (a solution containing HEPES-KOH, potassium acetate, magnesium acetate, calcium chloride, L-amino acid and/or dithiothreitol) can be used as extractant. The compositions and concentrations of each component in an extractant are known per se, and those employed in a method for producing a wheat embryo extract solution for cell-free protein synthesis may be adopted.

Embryo and a required amount of extractant for extraction are mixed, and the embryo is fragmented in the presence of the extractant. The amount of extractant is usually 0.1 mL or more, preferably 0.5 mL or more, more preferably 1 mL or more to 1 g of embryo before washing. The upper limit of amount of extractant is not limited in particular, but usually 10 mL or less, and preferably 5 mL or less to 1 g embryo before washing. Further, about the embryo to be fragmented, the frozen as usual and the non-frozen may be used, and the non-frozen is preferred for use.

About the method for fragmentation, a conventionally known method can be employed as a grinding method such as milling and homogenating, and a method for fragmenting embryo by giving impact or chopping is preferred, and that method was developed by the inventors (Patent Publication WO 03/064671). Herein, “fragmenting by giving impact or chopping” means to break a plant embryo in a condition allowing the break of organelles such as cell nucleus, mitochondria and chloroplast; cell membrane and wall and the like in a plant embryo to be limited to the minimal extent comparing to conventional milling or homogenating methods.

An apparatus and method for use in fragmentation described is not limited in particular as long as they meet the above conditions and, for example, an apparatus having high speed rotary blade matter such as Waring blender is preferred for use. The rotary speed of blade matter is usually 1000 rpm or more, and preferably 5000 rpm or more, or usually 30000 rpm or less, and preferably 25000 rpm or less. Rotation period of blade matter is usually 5 seconds or more, and preferably 10 seconds or more. The upper limit of rotation period is not limited in particular, but usually 10 minutes or less, and preferably 5 minutes or less. The temperature at fragmentation is preferably within a range from 10° C. down to still an operable temperature, and particularly around 4° C. is suitable.

Fragmenting embryo by giving impact or chopping like this allows the cell nucleus and cell wall of embryo to break but not completely and at least a fraction thereof escapes from breaking and remains. Therefore, organelles such as cell nucleus, cell membrane and wall of embryo are not broken than need be, so that the contamination of impurities such as DNA and lipid contained therein is slight, thus cytoplasm-localized RNA, ribosome and the like necessary for protein synthesis can be extracted from embryo efficiently with a high purity.

According to this method, a wheat embryo extract solution can be obtained efficiently because the conventional step of grinding plant embryo and step of obtaining wheat embryo extract solution by mixing the ground plant embryo and an extractant can be conducted simultaneously as one step. The method described above is sometimes referred to as “blender method.”

Such fragmentation of plant embryo, in particular fragmentation by giving impact or chopping is preferred to be conducted in the presence of extractant, but the extractant can also be added after fragmentation.

Then, wheat embryo extract solution can be collected by centrifugation and the like and purified by gelfiltration and the like, obtaining a wheat embryo extract solution. Gelfiltration can be conducted, for example, using a gelfiltration apparatus which has been equilibrated with an appropriate solution beforehand. The compositions and concentrations of each component in gelfiltration solution is known per se, and those for use in a method for producing a wheat embryo extract solution for cell-free protein synthesis (for example, a solvent containing HEPES-KOH, potassium acetate, magnesium acetate, dithiothreitol or L-amino acid) may be adopted.

Preferably, thus obtained cell extract has extremely decreased RNase and phosphatase activities.

Sometimes, solution containing embryo extract after gelfiltration may be contaminated with microorganisms, in particular spores of filamentous bacteria (mold) and the like, so that the elimination of these microorganisms is preferred. In particular, as the growing of microorganisms can be seen during the long-term (more than a day) reaction of cell-free protein synthesis, blocking their growing is important. A means for eliminating microorganisms is not limited in particular, but using a filter-sterilization filter is preferred. The pore size of filter is not limited in particular as long as suspected contaminating microorganisms can be removed, and usually from 0.1 to 1 μm is suitable, and preferably from 0.2 to 0.5 μm. In fact, the spore size of smaller class-sized Bacillus subtilis is 0.5 μm×1 μm, thereby using 0.20 μm filter (for example, Minisart™ from Sartorius) is effective to remove spores. On the occasion of filtration, it is preferred to conduct filtration using filters firstly having a bigger pore size, and then having a pore size capable of removing suspected contaminating microorganisms.

In thus obtained cell extract, substances inhibiting protein synthesis functions (substances affecting mRNA, tRNA, protein translation factors, ribosome and the like and inhibiting their functions, such as tritin, thionine and RNase), which a source material of wheat embryo itself contains or holds, have almost completely been removed. Namely, endosperm where these inhibitors are localized have almost completely been removed, purifying the cell extract. The level of removal of endosperm can be assessed by monitoring tritin activity contaminating in a wheat embryo extract, in other word, activity of deadenylating ribosome. When ribosome is not substantially deadenylated, it means that there is no contaminating component derived from endosperm in the embryo extract, thus it can be judged that endosperm is almost completely removed and the extract is purified. The level that ribosome is not substantially deadenylated means that the deadenylation rate of ribosomal is less than 7%, and preferably 1% or less.

Using an embryo extract like this as source material and for further “controlling ATP-mediated sugar phosphorylation pathway” described above, the present invention conducts a treatment for preparing a cell extract for cell-free protein synthesis wherein sugars, phosphorylated sugars, sugar phosphorylation enzymes, glycolytic enzymes and the like are controlled. Overview of treatment steps are as follows.

A centrifuged supernatant is obtained by centrifuging embryo extract solution as a source material at from 20,000 to 40,000 G, preferably from 25,000 to 35,000 G, and more preferably 30,000 G. At that time, as a precipitation auxiliary agent, adding inorganic carrier is more preferred for separating a precipitate from a supernatant. In this precipitate, complexes between enzymes like glycosidase and calcium are contained. The removal of glycosidase beforehand helps to minimize the production of glucose from starch. Examples of suitable inorganic carriers include bentonite, activated carbon, silica gel, sea sand and the like. Introduction of this inorganic carrier almost completely prevents the supernatant from being contaminated with precipitates. If a precipitation auxiliary agent is not added on centrifugation, an insoluble slurry would be generated on the upper part of the precipitate, and an extract solution prepared from an S-30 fraction contaminated with it would show a lower activity of protein synthesis. Therefore, on collecting an S-30 fraction from a centrifuge tube after centrifugation, a meticulous attention is needed to avoid contamination.

The obtained supernatant by centrifugation is changed to a translation reaction solution by conducting solution exchange through gelfiltration or adding required components, and a low molecular fraction is eliminated from the solution by molecular weight fractionation with 10,000 Da molecular weight cutoff. Alternatively, substances having a molecular weight of 10,000 Da or more can be collected by molecular weight fractionation. It is preferred to repeat this fractionation multiple times and substantially remove especially substances having a molecular weight of 10,000 Da or less. The specific number of multiple times is from 1 to 10, preferably from 2 to 9, more preferably from 3 to 8, and most preferably from 4 to 7. Sugars and phosphorylated sugars of thus prepared cell extract are substantially decreased to 6 mM or less (as the concentration of glucose in the extract solution having an absorbance of 200 OD/mL at 260 nm). The extract solution having decreased concentration of glucose obtained like this has an unprecedentedly high capacity of cell-free protein synthesis.

The cell extract of the present invention wherein ATP-mediated sugar phosphorylation pathway intrinsic to a cell has been controlled (in other words, cell-intrinsic mechanism for inhibition of translation has been eliminated) prepared in this way can be used as it is, while even such removal has not been completely conducted, if any one means of various inhibition and inactivation means described above is carried out, an unprecedentedly high capacity of cell-free protein synthesis can be achieved.

The cell extract of the present invention wherein ATP-mediated sugar phosphorylation pathway is controlled is also aiming a cell extract wherein at least one means selected from the followings has been introduced. Specific examples of those means are as described above.

1) substantially removing or inactivating phosphorylated sugars,

2) substantially removing polysaccharides, oligosaccharides, disaccharides and monosaccharides,

3) substantially removing or inactivating glycolytic enzymes,

4) adding a glycolytic enzyme inhibitor,

5) substantially removing or inactivating phosphorylation enzymes,

6) adding a phosphorylation enzyme inhibitor.

Thus prepared cell extract will provide a cell-free protein synthesis method with an unprecedentedly high efficiency, and moreover with a use of cell-free protein synthesis system using this cell extract, a high utility can be achieved in various analytic and screening methods. Further, with a reagent kit for use in a cell-free protein synthesis system containing a cell extract provided by the present invention, unprecedented effects on protein synthesis can be achieved as a cell-free protein synthesis means.

A translation reaction solution is adjusted by adding components necessary for protein synthesis to a solution containing a cell extract prepared as stated above. Alternatively, a cell extract is passed through a Sephadex G25 column equilibrated with a solution containing component necessary for protein synthesis, and so an eluent is changed to a translation reaction solution. The components necessary for protein synthesis are as follows: nuclease inhibitors, various ions, amino acids as substrate, energy sources and the like (hereinafter, they are sometimes referred to as “additives of translation reaction solution”), and mRNA encoding a particular protein as a translation template, in addition, if desired, stabilizer containing at least one kind of component selected from the group consisting of inositol, trehalose, mannitol and sucrose-epichlorohydrin copolymer, and the like. Addition concentrations of each component can be achieved by a compounding ratio well known per se.

Additives of translation reaction solution include, in particular, amino acids as substrate, energy sources, various ions, buffer, ATP-regenerator, nuclease inhibitor, tRNA, reducing agent, polyethylene glycol, 3′,5′-cAMP, folate, antimicrobial agent and the like. Further, it is preferred that respective concentration becomes as follows: ATP is from 100 μM to 0.5 mM, GTP is from 25 μM to 1 mM, and twenty kinds of amino acids are respectively from 25 μM to 5 mM. They can be selected as appropriate and used in combination depending on a translation reaction pathway. Specifically, when a wheat embryo extract solution is used, an example of a solution containing a cell extract is prepared as follows: 30 mM HEPES-KOH (pH7.8), 100 mM potassium acetate, 2.7 mM magnesium acetate, 0.4 mM spermidine (supplied by Nacalai Tesque), each 0.3 mM twenty kinds of L-amino acids, 4 mM dithiothreitol, 1.2 mM ATP (supplied by Wako Pure Chemical Industries, Ltd.), 0.25 mM GTP (supplied by Wako Pure Chemical Industries, Ltd.), 16 mM phosphocreatine (supplied by Wako Pure Chemical Industries, Ltd.), 40 μg/ml creatine kinase (supplied by Roche), 0.005% sodium azide are added, and after dissolving them well, an appropriate amount of translation template mRNA is added thereto.

Herein, mRNA is any one as long as it has a structure in which the coding region of a protein to be synthesized by a cell-free protein synthesis system is linked downstream to a sequence which an appropriate RNA polymerase can recognize and further a sequence having a translation-activating function. The sequence which RNA polymerase can recognize includes T3 or T7 RNA polymerase promoter and the like. When a reagent for a cell-free protein synthesis of the present invention is used to make a protein chip, library and the like, it is selected as appropriate depending on the respective object. Further, as an sequence to facilitate a translation activity in a cell-free protein synthesis system, a sequence having a structure in which Q sequence, E01 sequence (described in Patent Publication WO 03/056009, SEQ ID NO: 136) or the like is linked to 5′ upstream of coding sequence is preferably used.

The present invention is explained in detail below with reference to examples, but these examples are not intended to limit the scope of the present invention.

### EXAMPLE 1

Preparation of Highly Functionalized Wheat Embryo Extract Solution Using Precipitation Auxiliary Agent

(1) Preparation of Wheat Embryo

Chihoku wheat seeds grown in Hokkaido or Chikugoizumi seeds grown in Ehime were supplied to a mill (from Fritsch: Rotor Speed Mill pulverisette 14) at the rate of 100 g/min, and ground gently at the rotating speed of 8,000 rpm. A fraction containing a germinable embryo was collected with a sieve (mesh size from 0.7 to 1.00 mm), and then subjected to flotation using a mixture of carbon tetrachloride and cyclohexane (at the volume ratio of carbon tetrachloride:cyclohexane=2.4:1) to collect a floating fraction containing a germinable embryo, dried at a room temperature to remove organic solvents, and then blown at a room temperature to remove impurities such as contaminating seed coats and the like to obtain a crude embryo fraction.

Next, using a belt type color sorter BLM-300K (supplier: Anzai Manufacturing Co., Ltd., distributor: Anzai Corporation Ltd.), embryo was sorted out from a crude embryo fraction using color differences as below. This color sorter is an apparatus comprising: means to irradiate light on a crude embryo fraction, means to detect a reflected and/or transmitted light therefrom, means to compare detected values with a reference value, and means to sort out embryos either falling within or beyond the reference value and remove them.

A crude embryo fraction was supplied on a beige-colored belt of the color sorter at 1000-5000 grains/cm², which on the belt was irradiated with light from a fluorescent lamp, and reflected lights were detected. The rate of the conveyor belt was 50 m/min. As a photoreceiving sensor, a monochrome CCD line sensor (2048 pixel) was used.

Firstly, to remove components having a darker color than that of embryo (seed coat etc.), reference value was set up between luminances of embryo and seed coat, and those beyond the reference value were sucked and removed. Then, to sort out endosperm, the reference value was set up between luminances of embryo and endosperm, and those beyond reference value was sucked and removed. Sucking was conducted using 30 sucking nozzles installed at a position approximately 1 cm above the conveyor belt (sucking nozzles were lined at every 1 cm).

This step to sort out embryo was repeated until the purity of embryo (weight ratio of contained embryo to lg of random sample) became 98% or higher.

The obtained wheat embryo fraction was suspended in distilled water at 4° C. and washed by an ultrasonic cleaner until wash solution became clear. Then, the fraction was suspended in a 0.5 vol % solution of Nonidet P40 (supplied by Nacalai Tesque) and washed by the ultrasonic cleaner until wash solution became clear, obtaining a wheat embryo. An extractant (80 mM HEPES-KOH, pH7.8, 200 mM potassium acetate, 10 mM magnesium acetate, 8 mM dithiothreitol, 4 mM calcium chloride, and each 0.6 mM twenty kinds of L-amino acids) was added, in double volume to the wet weight of the collected embryo, and a limited grinding was conducted on the embryo using Waring blender for three times, each time at 5,000 to 20,000 rpm for 30 seconds.

(2) Preparation of S-30 Fraction Using Precipitation Auxiliary Agent

To the obtained homogenate described above, 20 wt % sea sand or swollen Sephadex G25 particle was added and stirred. Before adding to the homogenate, sea sand was pretreated as follows: washed with water→washed with 5 vol. of 0.1 N NaOH or KOH→washed with water→washed with 0.1 N HCl→washed with water→heated to 100 to 120° C. for RNase inactivation, and then subjected to drying.

The homogenate mixed with sea sand was centrifuged twice at 30,000×g for 30 minutes, then once again for 12 minutes to obtain a translucent centrifuged supernatant (S-30 fraction). When Sea sand or Sephadex particle was not added before centrifugation, insoluble slurry was generated on the upper part of the precipitate, and an extract solution prepared from S-30 fraction contaminated with the slurry showed a lower activity in protein synthesis. The obtained S-30 fraction was subjected to Sephadex G25, which had been equilibrated with an eluent (40 mMHEPES-KOH, pH7.8, 200 mM potassium acetate, 10 mM magnesium acetate, 4 mM DTT), subjected to gelfiltration, preparing an embryo extract solution from which low molecular substances having molecular weight of 1000 Da or less had been eliminated.

(3) Protein Synthesis

To an embryo extract solution, components necessary for translation were added and adjusted to prepare a translation reaction solution (30 mM HEPES-KOH, pH7.8, 100 mM potassium acetate, 2.7 mM magnesium acetate, 1.2 mM ATP, 0.25 mM GTP, 0.4 mM spermidine, 16 mM phosphocreatine, 40 μg/ml creatine kinase, 4 mM dithiothreitol, each 0.3 mM 19 kinds of leucine-lacking L-amino acids, and 0.005% sodium azide). The concentration of the embryo extract solution was 40 OD 260 nm per 1 ml of translation reaction solution). To this translation reaction solution, mRNA encoding dihydrofolate reductase (DHFR) (0.32 mg/ml) and ¹⁴C-leucine were added, and protein synthesis was conducted at 26° C. through a batch method. The amount of synthesized protein was determined by measuring radioactivity of ¹⁴C-labeled leucine incorporated into an acid-insoluble fraction as follows: a 3MM Whatman filter was blotted with 5 pL of the reaction solution, and the filter was dipped into an ice-cold 10% TCA (trichloroacetic acid) for an hour, and then boiled in 5% TCA solution for 10 minutes. This filter was recovered and from which TCA and water were removed using ethanol and ether (50:50 vol), dried, and then radioactivity incorporated into a heat-TCA-insoluble fraction was measured by a liquid scintillation counter (toluene scintillator).

FIG. 1A shows effects of a precipitation auxiliary agent. ∘ represents the amount of synthesized protein using S-30 fraction which had been prepared by a conventional method without using a precipitation auxiliary agent. ● (large) represents the amount of synthesized protein using S-30 fraction which had been prepared using sea sand as a precipitation auxiliary agent. Coprecipitation operation for insoluble matters using sea sand during centrifugation led to an increase in the activity of protein synthesis by 20 to 30% (FIG. 1A). Therefore, it was believed that by the centrifugation in the absence of sea sand, S-30 collected from the supernatant was contaminated with the precipitate capable of inhibiting protein synthesis. It was demonstrated that these effects could be obtained by anything other than sea sand as long as it has coprecipitation effects, and so could a swollen commercially available Sephadex particle (G25) (●: small filled circle).

### EXAMPLE 2

Removal of Inhibitors through Amicon Ultramembrane Filtration

The reaction solution for protein synthesis (besides extract solution, comprising components necessary for protein synthesis other than mRNA respectively at optimal concentration) prepared using a precipitation auxiliary agent as indicated above was passed through Amicon Ultra-15 centrifugal filter device (15 ml, 10K NMWL, supplied by MILLIPORE) with a molecular weight cutoff of 10,000 to further eliminate substances having low molecular weight below 10,000 Da, preparing protein synthesis solution. This filtration was repeated six times. This protein synthesis solution is hereinafter referred to as highly functionalized protein synthesis solution. As shown in FIG. 1B (●), by this filtration, synthesis reaction, which had been terminated in about 2 hours, became sustained at least in three hours, and translation activity was increased as well (● in FIG. 1B were compared with large and small ● in FIG. 1A). Therefore, it was demonstrated that Amicon ultra-centrifugal filtration led to an increase in translation activity and also a significant improvement of the stability of translation reaction pathway. In addition, when the filtrate eliminated in the Amicon ultra-centrifugal filtration was returned to its reaction pathway, like the filtrate not subjected to Amicon ultra-centrifugal filtration, the activity of protein synthesis was decreased, the period of synthesis reaction shortened to for about two hours, and the reaction becoming unstable. Thereby, it was found that in this Amicon ultra-elimination fraction, there were intrinsic inhibitory factors having a molecular weight of 10,000 or less and capable of inhibiting and destabilizing the reaction of wheat embryo cell-free protein synthesis (∘ in FIG. 1B). Herein, a fact needed to pay attention again in the results of FIG. 1 is that the synthesis rate of reaction solution which had not been subjected to Amicon ultra-centrifugal filtration started to decrease one hour after the reaction started, and after about two hours, the activity of protein synthesis was terminated (∘ in FIG. 1A), while in the reaction solution subjected to Amicon ultra-centrifugal filtration, the reaction was sustained at least in 3 hours (● in FIG. 1B).

Next, destabilization of protein synthesis was further examined. Namely, samples obtained before and after Amicon ultra-centrifugal filtration as described above (but mRNA and ¹⁴C-leucine had not been contained), were incubated for three hours at 26° C. (hereinafter, described as preincubation), and then both reaction solutions were respectively passed through Sephadex G25 spin column which had been equilibrated with a translation solution (containing amino acids, energy sources, various ions and buffer) for changing to a fresh translation solution, and after that, mRNA and ¹⁴C-leucine were added to measure the activity of protein synthesis. In the sample not subjected to Amicon ultra-centrifugal filtration, the activity of protein synthesis was almost disappeared by preincubation (Δ in FIG. 1B), while in the reaction solution from which low molecules had been eliminated through Amicon ultramembrane, the activity was hardly decreased by preincubation (□ in FIG. 1B). Therefore, these results exhibited that the filtrate obtained using Amicon ultramembrane contained components (mechanism) which could cause irreversible inactivation to particular protein synthesis factors, so that a remarkable stabilization of a reaction solution could be achieved by eliminating inhibitory factors in an extract solution through a filtration operation using that membrane.

### EXAMPLE 3

Identification of Inhibitory Factors in Fraction to be Eliminated through Amicon Ultramembrane

A filtrate obtained by elimination of Amicon ultramembrane filtration was developed using a thin layer chromatography (TLC (silica gel plate (10 cm×10 cm), supplied by Merck)) with a solution having a volume ratio of methanol:concentrated ammonia water (22%)=1:1 (spreading was conducted at room temperature for 5 minutes)as a developing solvent, and separated blots were detected by oxidization with concentrated sulfuric acid (FIG. 2A). At the same time, standard substance was developed, and from its position (RF value), it was revealed that this filtrate comprised glucose, glucose 1-phosphate (or mixture with glucose 6-phosphate), fructose phosphate (mixture of fructose 6-phosphate and fructose 1,6-diphosphate), raffinose, sucrose, galactose and a yellow substance (Y in FIG. 2A), and among them, the concentration of glucose was high. When this filtrate before fractionation was added to a highly functionalized protein synthesis solution in an equivalent amount, a strong inhibition of protein synthesis was confirmed (∘ in FIG. 1B). Then, each component was extracted separately and then added them back in an equivalent amount which was calculated from the volume in each step of experiment operation, confirming that glucose and sucrose (large ●: glucose added, small ●: sucrose added, in FIG. 2B) had strong inhibitory and destabilization effects on protein, and raffinose and yellow substance did not show inhibitory effects (data not shown). As shown from the kinetics of reaction, in an extract solution filtered through Amicon ultramembrane, as exhibited in FIG. 1B, protein synthesis was sustained at least for three hours, however when the solution was added with sucrose and glucose which was derived from the filtrate, a protein synthesis capacity was decreased already in an hour after the reaction and the reaction was terminated in two hours. As a result, a yield of synthesized product was decreased. The presence of raffinose, sucrose, glucose and phosphorylated sugar in the filtrate detected in FIG. 2A were identified and confirmed by nuclear magnetic resonance measurement.

Using another several saccharides of standard products, their effects on protein synthesis were examined (FIG. 2C). Each sugar was added to the synthetic reaction pathway to a final concentration of 0.5 mM, and all of D-glucose (Δ-Δ), fructose (□-□), galactose (⋆-⋆), glucose-6-phosphate (small ▴-▴) and sucrose (★-★) showed strong protein synthesis inhibitory effects, and reaction durations were shortened, thus the destabilization was confirmed. These effects were also shown in 0.3 mM glucose (Δ-Δ). Values of sucrose (★-★), galactose (⋆-⋆), glucose-6-phosphate (small ▴-▴), 0.3 mM glucose (Δ-Δ) were about the same (in the figure, they were intentionally illustrated apart).

Next, to confirm the destabilization phenomena of protein synthesis system resulting from the addition glucose and preincubation, the system was incubated in the presence of 0.5 mM glucose (the system contained neither mRNA or ¹⁴C-leucine) and the stability was examined in the same manner as explained in FIG. 1B. As shown in ▴ (large) in FIG. 2C, it can be seen that incubation in the presence of 0.5 mM glucose irreversibly inactivates a protein synthesis capacity. Therefore, it was exhibited that at least one of factors found in a wheat embryo extract solution (S-30) and causing the inhibition and destabilization of protein described above was glucose. Though the inhibition was also confirmed by adding galactose, the addition of L-glucose, which was a stereoisomer of glucose and never recognized by biological enzymes, showed no inhibitory effects even in the amount of 0.5 mM (large ●-● in FIG. 2C). Phosphoenolpyruvic acid (small ●-● in FIG. 2C) and pyruvic acid (medium ●-● in FIG. 2C) which are the end side metabolites of glycolytic pathway, showed no inhibitory effects on protein synthesis.

### EXAMPLE 4

Determination of Concentration of Glucose

As a method for pre-treatment to measure the concentration of total glucose (free and phosphorylate types (mainly glucose 1-phosphate)), samples were boiled under the 1.0N hydrochloric acid for 5 minutes, neutralized with sodium hydroxide, and subjected to both measurements, Glucose oxidase/Mutarotase method and 0-toluidine-borate method.

As shown in Table 1, from the determination by Glucose oxidase/Mutarotase method, a high concentration of over 30 mM total glucose exists in S-30 fraction (200 OD 260 nm) (the concentration of free glucose was 7.8 mM which was measured using a sample not treated with acid and heat), but it can be decreased by passing it through Sephadex G25 column or Amicon ultramembrane (in the table: abbreviated as A.U.). A concentration of total glucose was decreased to 5.5 mM by gelfiltration with Sephadex G25 column. Further, the concentration of total glucose was decreased in a stepwise fashion by repeating centrifugal filtration using Amicon ultramembrane, and in a sample after repeating that filtration operation six times, the concentration was decreased to 3.4 mM (the concentration of free glucose was 0.4 mM). While, from the determination of those fractions by O-toluidine-borate method, total aldohexose and aldopentose of over 72.2 mM, which was over 2.3-fold that measured by enzyme method, were detected in S-30 fraction. It was found that the concentration of total glucose was decreased to 6.9 mM by repeating gelfiltration operation six times. At that time, the concentration of free glucose was 0.4 mM. Without performing centrifugal filtration operation, elimination of glucose from a high-volume extract solution can be achieved using VIVAFLOW enriched membrane (supplied by Sartorius, VIVAFLOW 50, molecular weight cutoff value of 10,000 Da) and Peristaltic Pump. It was exhibited that by using VIVAFLOW enriched membrane, an extract for wheat embryo cell-free protein synthesis solution having the similar performance as that obtained by using Amicon ultramembrane would be produced. In this method, the filtration of concentrating operation was repeated six times with substrate solution having a volume equivalent to that of extract solution. As a result, the concentration of total glucose became 3 mM in which that of free glucose became 0.4 mM, thus it was exhibited that an extract for wheat embryo cell-free protein synthesis solution having the similar performance as that obtained by using Amicon ultramembrane could be produced. By using Sephadex G25 column and VIVAFLOW enriched membrane, further elimination of monosaccharides can be expected. S-30 fraction subjected to gelfiltration by Sephadex G25 column was further subjected to concentrating operation six times as described above using VIVAFLOW enriched membrane, thereby remarkable elimination effects were confirmed in total glucose, total aldohexose and aldopentose. Specifically, the concentration of total glucose became 0.6 mM, and that of free glucose became 0.3 mM.

Using these embryo extracts, following the method described in Example 1, and under a condition where the concentration of extract solution was 40 OD 260 nm, protein synthesis was conducted and their synthesis activities were compared. The activities of protein synthesis were determined by measuring radioactivities of ¹⁴C/5 μL reaction solution, wherein ¹⁴C had been incorporated into a heat-acid-insoluble fraction after a three hour-long reaction. As gelfiltration operation proceeded, the concentration of glucose in an extract solution was decreased, and completely corresponding to that, the activity of protein synthesis was increased, and simultaneously an extremely stabilized reaction solution for protein synthesis was produced.

TABLE 1

| | Glucose | | | |
|---|---|---|---|---|
| | (mM/200A 260) | | | Activity of |
| | Glucose oxidase | | O-toluidine | protein synthesis |
| Sample | method | | method | dpm |
| S-30 | 31.2 | (7.8) | 72.2 | Not measured |
| G-25 | 5.5 | | 12.8 | 3111 |

A.U. filtration (repeats)

| | | | | |
|---|---|---|---|---|
| (1) | 16.4 | | 43.9 | 2145 |
| (2) | 13.5 | | 27.2 | 2574 |
| (3) | 10.9 | | 19.8 | 2772 |
| (4) | 7.1 | | 14.9 | 2970 |
| (5) | 5.3 | | 10.2 | 3135 |
| (6) | 3.4 | (0.4) | 6.9 | 3234 |
| (7) | 3.3 | | 6.1 | 3300 |
| VIVAFLOW | 3.0 | (0.4) | 3.4 | 3313 |
| enriched membrane | | | | |

S-30 → VIVAFLOW enriched membrane

| | | | | |
|---|---|---|---|---|
| | 0.5 | (0.3) | 0.6 | 3824 |
| Dialysis | 8.7 | (0.8) | | 2640 |
| incubation treatment | | | | |

O-toluidine-borate method (Wako Pure Chemical Industries, Ltd. Code No. 439-90901)
Glucose oxidase/Mutarotase (Wako Pure Chemical Industries, Ltd. Code No. 273-13901)
Numbers in parentheses represent the concentration of free glucose.

### EXAMPLE 5

Decreased ATP-Dependent Inhibition and Destabilization of Protein Synthesis Reaction

The relation between hexose such as glucose present in reaction solution and the phenomenon of decreased concentration of ATP was experimentally examined. Namely, temporal changes of the concentration of glucose during protein synthesis reaction were determined. When an energy regenerator is present in protein synthesis reaction, the concentration of remaining glucose after the six times filtration through Amicon ultramembrane is almost metabolized by an hour-long incubation. (∘ in FIG. 3A). While, in the absence of an energy regenerator (in the absence of creatine kinase, and ATP is only 1.2 mM which was added at the beginning of the reaction), the amount of glucose decreased by metabolism is small (● in FIG. 3A). Namely, from the facts (1) supply of ATP is essential for the metabolism of glucose, and (2) the presence of phosphorylated sugar found in FIG. 2A, it was believed that there could be a close relation between the consumption of ATP in a reaction solution for protein synthesis and phosphorylation of glycolytic pathway.

Next, to study the relation between decrease in concentration of ATP and the inhibition and destabilization mechanism of protein synthesis reaction, temporal changes of concentration of ATP under incubation was measured in the present and absence of glucose. Experiment was conducted on chromatography using a cellulose thin layer plate (Avicel: purchased from Funakoshi Co., Ltd) and a developing solvent of isobutyric acid:0.5 M ammonia (5:3). Using a synthesis system prepared with an extract solution treated through Amicon ultramembrane six times, protein synthesis reaction was conducted with or without adding glucose (1 mM), and to samples fractionated with time, an equivalent amount of cold ethanol was added, then supernatants obtained from centrifugation at 10,000×g were used to fractionate nucleotides on a thin layer plate. Using standard nucleotides from a conventional method as a fractionation marker, ATP was extracted from fractions, which was scraped out from fluorescent blots visualized with irradiation of ultraviolet ray, and the values of ultraviolet absorption at 260 nm were measured, thereby temporary changes of concentration of ATP in a reaction solution were determined. In an experiment where glucose was not added, as reaction proceeded, the concentration of ATP in the reaction solution was decreased (large ● in FIG. 3B) and in three hours after the reaction, decreased to around 60%. Kinetics of this decrease in concentration of ATP is well consistent with a decrease in the concentration of glucose (● in FIG. 3A). Further, they are well consistent with the kinetics of a protein synthesis reaction using an extract solution where sugar had been reduced using Amicon ultramembrane exhibited in FIG. 1 and 2, and the like. Though data are not shown, when protein synthesis reaction was conducted using an extract solution of 25 OD 260 nm, a decrease in the concentration of ATP was hardly shown until four hours after reaction, and along with it, the protein synthesis reaction was sustained linearly almost for four hours.

In FIG. 3B, the result of glucose-added reaction is shown. In a reaction added with 1 mM commercially available D-glucose (combined with intrinsic glucose, final concentration was 1.082 mM), almost all ATP in the reaction solution was consumed in an hour (∘ in FIG. 3B), while in the case of adding 1 mM L-glucose and similar to the case where sugar was not added, nearly 50% ATP was remained (small ● in FIG. 3B), such temporary changes in concentration of ATP are also well consistent with the kinetics of protein synthesis reaction exhibited in FIG. 2C. Though Data are not shown, the increase in the concentration of AMP/ADP in accordance with the decrease in the concentration of ATP was confirmed as blots on a thin layer chromatography plate. From these results, it was found that hexose such as glucose and fructose in reaction solution was directly involved in the consumption of ATP as substrate.

### EXAMPLE 6

Effects of AMP and GMP on the Activity of Protein Synthesis

The activities of protein synthesis were measured when adding a high concentration of AMP or GMP, and AMP and GMP simultaneously, in addition to 1.26 mM ATP and 0.25 mM GTP, which were contained in a usual cell-free reaction solution for protein synthesis. For the control, protein synthesis was conducted using an extract having a concentration of 40 OD 260 nm which was obtained by six times filtration through Amicon ultramembrane (large ● in FIG. 4). To this protein synthesis reaction, 0.5 mM AMPand0.25 mM GMP were added, however inhibition of protein synthesis was not exhibited (small ● in FIG. 4). Though not shown in figures, inhibition of protein synthesis was not exhibited by the addition of respective 0.5 mM AMP or 0.25 mM GMP alone. Therefore, it was seen that the inhibition of protein synthesis reaction was not caused by the accumulation of byproducts AMP and GMP in the system. Adding 0.5 mM fructose, sucrose and galactose also decreased the concentration of ATP in system below the detection limit within an hour after reaction (data not shown). These results suggest that the decrease in the concentration of ATP irreversibly inactivates protein synthesis factors through a certain mechanism.

### BRIEF DESCRIPTION OF THE DRAWING

FIG. 1A shows effects of adding a precipitation auxiliary agent on centrifugation in preparation of S-30 fraction. ∘-∘ represents the activity values of protein synthesis of S-30 obtained without adding a precipitation auxiliary agent, and ●-● (large) represents those values adding with sea sand, and ●-● (small) with swollen Sephadex G25 particle with an extract solution, as precipitation auxiliary agents.

FIG. 1B shows that an extract solution with a high activity can be obtained by filtration through Amicon ultramembrane. The following represent the activities values of protein synthesis: ●-● with S-30 prepared using sea sand as a precipitation auxiliary agent, and then subjected to Sephadex G25, and then to six times filtration through Amicon ultramembrane, ∘-∘ with a sample which was filtered through Amicon ultramembrane, and then the concentrated filtrate was added back in an equivalent amount. Δ-Δ with an extract solution not subjected to filtration through Amicon ultramembrane but preincubated, □-□ with an extract solution through Amicon ultramembrane filtration and the extract solution was preincubated.

FIG. 2A shows thin layer chromatogram of saccharide components in the filtrate obtained through Amicon ultramembrane. Concentrated sulfuric acid-developed color blots were transferred.

FIG. 2B shows the inhibition of protein synthesis reaction by sucrose and glucose in the filtrate. Fractionated sucrose and glucose in FIG. 2A were isolated and respectively added to a protein synthesis reaction system, and examined on their inhibitory effects on synthesis by the method explained in FIG. 1. The activity values of protein synthesis were shown below: ∘-∘: saccharide was not added (control), large ●-●: glucose added, small ●-●: sucrose added.

FIG. 2C shows inhibitoryeffects onproteinsynthesis by standard sugar molecular species. The activity values of protein synthesis when adding the followings were shown, and final concentration of each is 0.5 mM; large ●-●:L-glucose, small ●-●: phosphoenolpyruvic acid, medium ●-●: pyruvic acid, Δ-Δ: D-glucose, □-□: fructose, ⋆-⋆: galactose, ★-★: sucrose, small ▴-▴: glucose-6-phosphate, Δ-Δ: 0.3 mM glucose. ∘-∘ represents control experiment without adding sugar. Large ▴-▴represents the activity value of protein synthesis by cell extract solution preincubated in the presence of 0.5 mM D-glucose.

FIG. 3A and FIG. 3B show a decrease in the concentration of ATP accompanying the metabolism of glucose during protein synthesis reaction. FIG. 3A shows temporary changes in concentration of glucose in the presence of creatine kinase (∘-∘), or in the absence of that (●-●). The initial concentration of reaction was 0.082 mM (100%). FIG. 3B shows changes in concentration of ATP associated with usual protein synthesis reaction (containing creatine kinase). The results: large ●-●: glucose not added (usual protein synthesis reaction), ∘-∘: added with commercially available D-glucose 1 mM, small ●-●: added with non-metabolized L-glucose 1 mM.

FIG. 4 shows that AMP and GMP never inhibit wheat embryo cell-free protein synthesis reaction. Large ●-●: control experiment, small ●-●: AMP (0.5 mM) and GMP (0.25 mM) added.

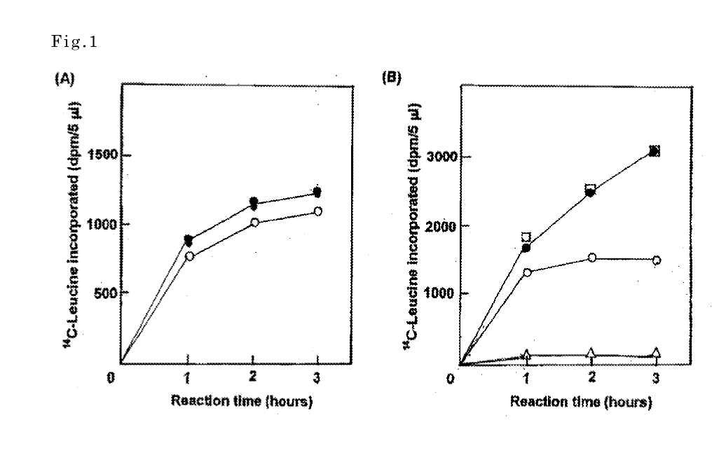

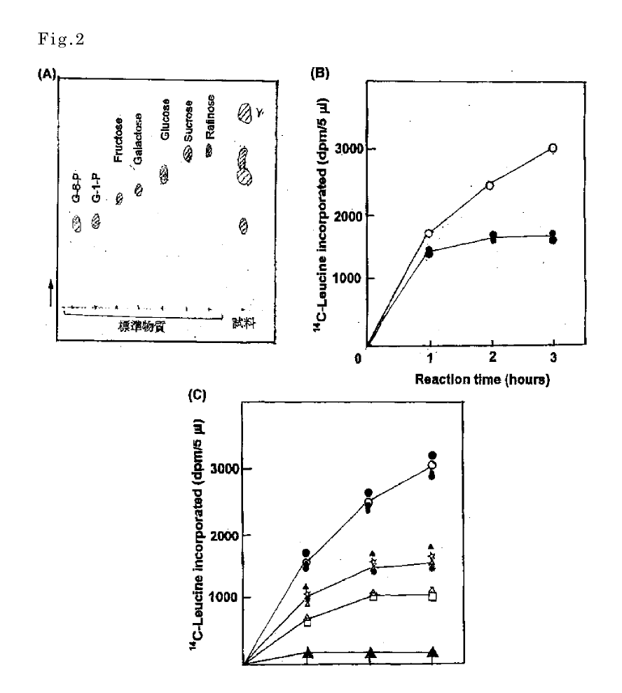

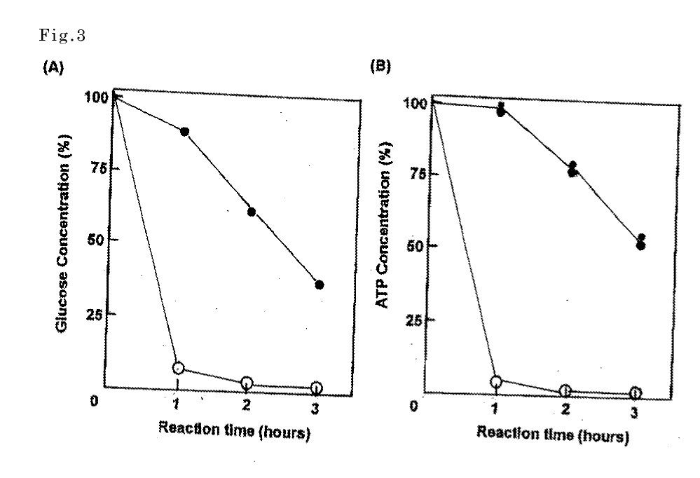

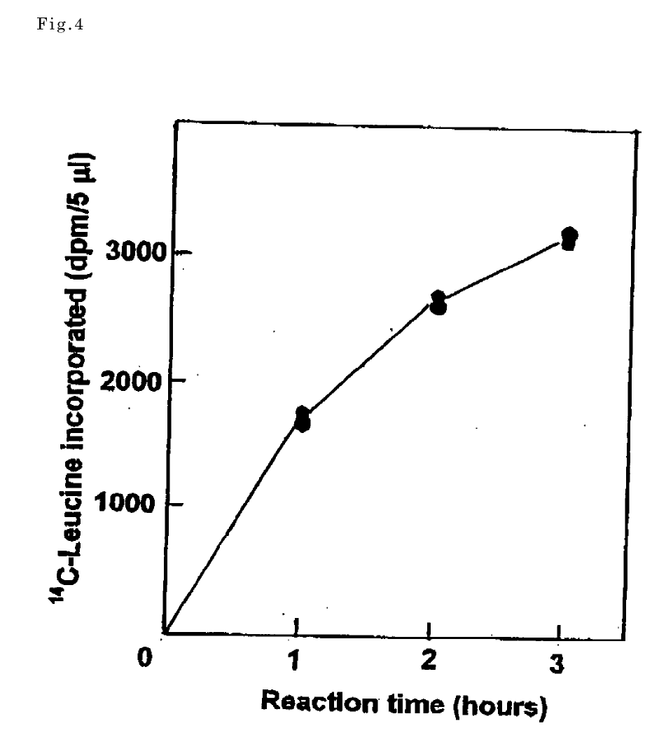

1. A method for preparing a cell extract for use in a cell-free protein synthesis means, comprising elimination of a cell-derived mechanism for inhibition of translation.

2. The method according to claim 1, wherein the elimination of a cell-derived mechanism for inhibition of translation is provided by controlling ATP-mediated sugar phosphorylation pathway.

3. The method according to claim claim 1, wherein the cell-derived mechanism for inhibition of translation is an embryo cell-intrinsic inducible system of inhibition of protein synthesis.

4. The method according to claim 1, wherein a source of the cell extract is a wheat embryo extract in which contaminating endosperm components and low molecular protein synthesis inhibitors are substantially removed.

5. The method according to claim 1, wherein a source of the cell extract is E. coli, rabbit reticulocyte or insect cell extract.

6. The method according to claim 2, wherein the ATP-mediated sugar phosphorylation pathway is controlled by introducing at least one step selected from the followings:
1) removing monosaccharides,
2) removing phosphorylated sugars,
3) controlling production of monosaccharides from polysaccharides, and
4) controlling production of phosphorylated sugars from monosaccharides.

7. The method according to claim 6, wherein, in removing monosaccharides, the monosaccharide is a hexose.

8. The method according to claim 6, wherein the phosphorylated sugar is at least one selected from glucose 1-phosphate, fructose 1-phosphate, galactose 1-phosphate, glucose 1,6-diphosphate, fructose 1,6-diphosphate, galactose 1,6-diphosphate in removing phosphorylated sugars.

9. The method according to claim 6, wherein the monosaccharides and/or the phosphorylated sugars are removed by fractional elimination of molecular weight carried out by gelfiltration and/or with an ultrafiltration membrane.

10. The method according to claim 9, wherein the fractional elimination of molecular weight carried out by gel filtration and/or with an ultrafiltration membrane is repeated multiple times.

11. The method according to claim 6, wherein the production of monosaccharides from polysaccharides is controlled by controlling production of glucose from starch.

12. The method according to claim 11, wherein the production of monosaccharides from polysaccharides is controlled by introducing at least one step selected from the followings:
1) removing or inactivating glycolytic enzymes,
2) eliminating polysaccharides and/or oligosaccharides, and/or disaccharides, and
3) adding a glycolytic enzyme inhibitor.

13. The method according to claim 12, wherein a glycolytic enzyme is removed or inactivated by removing a complex between said glycolytic enzyme and calcium after forming the complex.

14. A method for preparing cell extract, wherein removal of a cell-derived glycolytic enzyme is introduced by adding at least one selected from bentonite, activated carbon, silica gel, Sephadex and sea sand to said cell extract as a precipitation auxiliary agent.

15. The method according to claim 6, wherein the production of phosphorylated sugars from monosaccharides is controlled by introducing at least one step selected from the followings:
1) introducing an inhibitor against a sugar phosphorylation enzyme,
2) removing or inactivating an sugar phosphorylation enzyme,
3) eliminating said production from glucose metabolic pathway by enzymatic degradation of a hexose,
4) inhibiting an enzymatic reaction of sugar phosphorylation by chemical or enzymological modification of a hexose,
5) enzymatically and/or chemically alternating and/or modifying a phosphorylation site of the sugar, so that a phosphate group cannot bind to said phosphorylation site of the sugar.

16. The method according to claim 7, wherein the hexose is glucose.

17. The method according to claim 16, wherein a concentration of glucose in the cell extract is 10 mM or less when a concentration of the cell extract is 200 OD 260 nm.

18. (canceled)

19. The cell extract for use in a cell-free protein synthesis means prepared by the method according to claim 1.

20. A cell extract for use in a cell-free protein synthesis means, wherein ATP-mediated sugar phosphorylation pathway is controlled.

21. The cell extract according to claim 20, wherein the ATP-mediated sugar phosphorylation pathway is controlled by introducing at least one step selected from the followings:
1) substantially removing or inactivating phosphorylated sugars,
2) substantially removing polysaccharides, oligosaccharides, disaccharides and monosaccharides,
3) substantially removing or inactivating glycolytic enzymes,
4) adding a glycolytic enzyme inhibitor,
5) substantially removing or inactivating phosphorylation enzymes,
6) adding a phosphorylation enzyme inhibitor,
7) enzymatically and/or chemically alternating and/or modifying a phosphorylation site of the sugar, so that a phosphate group cannot bind to said phosphorylation site of the sugar.

22-24. (canceled)
